(19) United States
(12) Patent Application Publication
(10) Pub. No.: US 2026/0337132 A1
(43) Pub. Date: Oct. 29, 2026

(54) ANTIPOLYMERANT COMPOSITIONS TO MITIGATE DEPOSITION OF ACIDIC BYPRODUCTS

(71) Applicant: ECOLAB USA Inc., St. Paul, MN (US)

(72) Inventors: David DIXON, Baton Rouge, LA (US); Kameswara VYAKARANAM, Sugar Land, TX (US); Bassam ALNASLEH, Sugar Land, TX (US); Vanessa MENDES-RIBEIRO, New Orleans, LA (US)

(73) Assignee: ECOLAB USA Inc., St. Paul, MN (US)

(21) Appl. No.: 19/468,551

(22) Filed: Feb. 3, 2026

Related U.S. Application Data
(60) Provisional application No. 63/754,264, filed on Feb. 5, 2025.

(51) Int. Cl. C07C 17/00; C07D 211/94

(52) U.S. Cl. CPC C07C 17/00; C07D 211/94

(57) ABSTRACT

The present disclosure provides compositions and methods for inhibiting or preventing fouling. A method of inhibiting polymerization of an ethylenically unsaturated monomer in a medium may include adding an effective amount of a composition to the medium. The composition includes a polymerization inhibitor and a polar aprotic solvent. The polymerization inhibitor may include (4-hydroxy-2,2,6,6-tetramethylpiperidin-1-yl)oxyl (HTEMPO), a derivative thereof, and/or a hydroxylamine thereof.

### TECHNICAL FIELD

The present disclosure generally relates to compositions and methods for preventing or inhibiting fouling. More particularly, the disclosure relates to compositions and methods for inhibiting polymerization of reactive monomers.

### BACKGROUND

In the light ends section of an ethylene dichloride (EDC) production plant, chloroprene concentrates in the light ends overhead and often polymerizes by free-radical polymerization, thereby causing fouling of trays and condensers. The lights column purifies EDC by removing volatile components, such as chloroform, chloroprene, and other chlorinated hydrocarbons, which helps decrease fouling in the cracking furnace downstream.

Some chlorinated hydrocarbons, such as chloroprene, are reactive and polymerize under the conditions present within the lights column and downstream equipment. Some producers chlorinate chloroprene, thereby producing high boiling products that do not polymerize. In cases where chloroprene is not chlorinated, it concentrates in the reflux of the lights column. If chloroprene is concentrated to ~10%, severe polychloroprene fouling can result, which fouls and plugs the condenser, reflux lines, and top of the lights column.

While polymerization inhibitors have been used in the prior art to attempt to control fouling, the points/locations at which the inhibitors have been added tend to become plugged with a mixture of solids derived from the reaction of the polymerization inhibitor with HCl and water, which are present in the EDC stream. These solids also caused under deposit corrosion, evident by the presence of iron chlorides.

### BRIEF SUMMARY

The present disclosure provides compositions and methods for preventing fouling/inhibiting polymerization of reactive monomers.

In some embodiments, the disclosure provides a method of inhibiting polymerization of an ethylenically unsaturated monomer in a medium, comprising adding an effective amount of a composition to the medium, the composition comprising a polymerization inhibitor and a polar aprotic solvent, wherein the polymerization inhibitor comprises (4-hydroxy-2,2,6,6-tetramethylpiperidin-1-yl)oxyl (HTEMPO), a derivative thereof, and/or a hydroxylamine thereof, and inhibiting polymerization of the monomer.

In certain embodiments, the present disclosure provides a solution, comprising a polymerization inhibitor, a polar aprotic solvent, and a reaction product of an acid and the polymerization inhibitor, wherein the polymerization inhibitor comprises HTEMPO, a derivative thereof, and/or a hydroxylamine thereof.

The foregoing has outlined rather broadly the features and technical advantages of the present disclosure in order that the detailed description that follows may be better understood. Additional features and advantages of the disclosure will be described hereinafter that form the subject of the claims of this application.

### DETAILED DESCRIPTION

Various embodiments are described below. The relationship and functioning of the various elements of the embodiments will be better understood in light of the following detailed description. However, elements and embodiments are not strictly limited to those explicitly described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2 electrons, according to Huckel's Rule.

“Cycloalkyl” refers to a cyclic alkyl substituent containing from, for example, about 3 to about 8 carbon atoms, such as from about 4 to about 7 carbon atoms or about 4 to 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups, such as methyl groups, ethyl groups, and the like.

“Heteroaryl” refers to a monocyclic or bicyclic 5- or 6-membered ring system, wherein the heteroaryl group is unsaturated and satisfies Huckel's rule. Non-limiting examples of heteroaryl groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (e.g., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino(-N(R_{A})(R_{B}), wherein R_{A} and R_{B} are independently hydrogen, alkyl, or aryl), halo (fluoro, chloro, bromo, or iodo), silyl, nitro (—NO₂), an ether (—ORA wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The terms “polymer,” “copolymer,” “polymerize,” “copolymerize,” and the like include not only polymers comprising two monomer residues and polymerization of two different monomers together, but also include (co)polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, organic, inorganic, and/or functionally modified. A polymer of the present disclosure can be in the form of a solution, a dry powder, a liquid, or a dispersion, for example.

The present disclosure provides compositions and methods for inhibiting fouling and/or preventing or mitigating deposition of acidic byproducts. The compositions disclosed herein are stable in acidic environments and thus do not lead to fouling that typically occurs with prior art antifoulants. The presently disclosed compositions and methods prevent or mitigate acidic byproducts from depositing on reactor vessels in plants where acidic streams are used.

In some embodiments, the compositions and methods disclosed herein prevent or mitigate the formation of deposits at chemical injection sites within reactive monomer plants, such as an EDC purification unit, thereby allowing for more effective and efficient fouling and corrosion control. In certain embodiments, the methods and compositions may decrease or eliminate fouling in the “lights” column of the EDC unit within a vinyl chloride monomer (VCM) plant, optionally at the point of chemical injection.

For example, application of certain prior art compositions causes immediate precipitation in the quill when contacted with the acidic stream and also causes corrosion, thereby blocking the slip stream presumably due to localized accumulation of solids and further soaking of water and HCl under the solids deposition. Therefore, the compositions and methods disclosed herein potentially affect two aspects of the plant—possible mitigation of corrosion and possible increased activity of the antipolymerant/polymerization inhibitor.

The compositions and methods disclosed herein allow for a higher chloroprene concentration, which allows less EDC to be lost to incineration, which is advantageous to plant producers. The compositions and methods decrease solution polymer fouling and increase EDC yield. Additionally, the compositions and methods decrease popcorn fouling, which could affect the heat exchangers.

The present inventors discovered that the use of polar aprotic solvents in compositions including a polymerization inhibitor, such as (4-hydroxy-2,2,6,6-tetramethylpiperidin-1-yl)oxyl (HTEMPO), and/or a derivative thereof, prevents the precipitation or deposition of byproducts in acidic media and also prevents or inhibits corrosion.

In some embodiments, the polymerization inhibitor comprises HTEMPO, a derivative thereof, and/or a hydroxylamine thereof.

As an illustrative, non-limiting example, the polymerization inhibitor of the present disclosure may comprise the following structure:

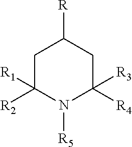

wherein each R₁, R₂, R₃ and R₄ is independently selected from a substituted or unsubstituted C₁-C₅₀ alkyl, alkenyl, or alkynyl group. As illustrative examples, each R₁, R₂, R₃ and R₄ may be independently selected from a substituted or unsubstituted C₁-C₄₀ alkyl, alkenyl, or alkynyl group, a substituted or unsubstituted C₁-C₃₀ alkyl, alkenyl, or alkynyl group, a substituted or unsubstituted C₁-C₂₀ alkyl, alkenyl, or alkynyl group, a substituted or unsubstituted C₁-C₁₀ alkyl, alkenyl, or alkynyl group, a substituted or unsubstituted C₁, C₂, C₃, C₄, C₅, C₆, C₇, C₈, C₉, or C₁₀ alkyl, alkenyl, or alkynyl group.

In some embodiments, at least one of R₁, R₂, R₃ and R₄ is a methyl group. In some embodiments, at least two of R₁, R₂, R₃ and R₄ are methyl groups. In some embodiments, at least three of R₁, R₂, R₃ and R₄ are methyl groups. In some embodiments, each of R₁, R₂, R₃ and R₄ is a methyl group. In certain embodiments, each of R₁, R₂, R₃ and R₄ is the same substituent or a different substituent.

The R variable may be selected from the group consisting of hydrogen, oxygen, alkyl (e.g., methyl, ethyl, propyl, butyl, etc.), hydroxyl, alkoxyl, amino, N-alkylamino, amido, N-alkylamido, carboxylate, and carboxyl.

The R₅ variable may be selected from hydrogen, hydroxyl, an oxygen free radical, a hydroxyl amine of HTEMPO, a hydroxyl amine of acetate TEMPO (2,2,6,6-tetramethylpiperidinyl-1-oxyl), and a reduced sebacate TEMPO.

Specific, non-limiting examples of polymerization inhibitors that can be used in accordance with compositions and methods disclosed herein include 4-hydroxy-2,2,6,6-tetramethyl piperidinol, 4-oxo-2,2,6,6-tetramethyl piperidinol, 4-methoxy-2,2,6,6-tetramethyl piperidinol, 4-acetate-2,2,6,6-tetramethyl piperidinol, 4-amino-2,2,6,6-tetramethyl piperidinol, 4-acetamido-2,2,6,6-tetramethyl piperidinol, 1,2,3,6-tetrahydro-2,2,6,6-tetramethyl piperidinol, bis(2,2,6,6-tetramethyl piperidinol) sebacate, and any combination thereof.

Additional examples include

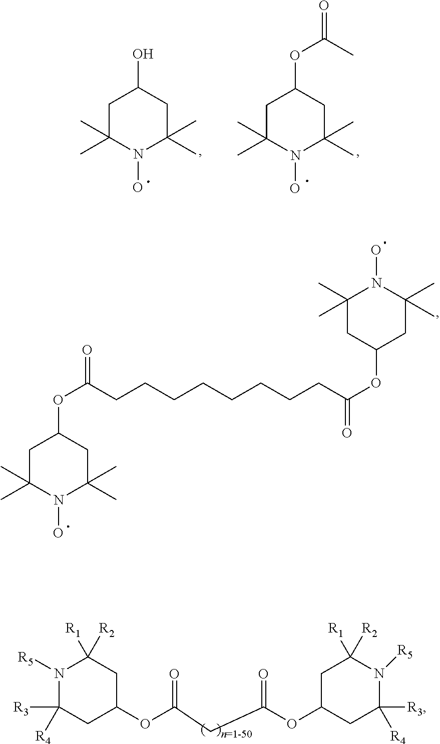

and any combination thereof, wherein each R₁, R₂, R₃ and R₄ variable is independently selected from a substituted or unsubstituted C₁-C₅₀ alkyl, alkenyl, or alkynyl group. As illustrative examples, each R₁, R₂, R₃ and R₄ may be independently selected from a substituted or unsubstituted C₁-C₄₀ alkyl, alkenyl, or alkynyl group, a substituted or unsubstituted C₁-C₃₀ alkyl, alkenyl, or alkynyl group, a substituted or unsubstituted C₁-C₂₀ alkyl, alkenyl, or alkynyl group, a substituted or unsubstituted C₁-C₁₀ alkyl, alkenyl, or alkynyl group, a substituted or unsubstituted C₁, C₂, C₃, C₄, C₅, C₆, C₇, C₈, C₉, or C₁₀ alkyl, alkenyl, or alkynyl group.

In some embodiments, at least one of R₁, R₂, R₃ and R₄ is a methyl group. In some embodiments, at least two of R₁, R₂, R₃ and R₄ are methyl groups. In some embodiments, at least three of R₁, R₂, R₃ and R₄ are methyl groups. In some embodiments, each of R₁, R₂, R₃ and R₄ is a methyl group. In certain embodiments, each of R₁, R₂, R₃ and R₄ is the same substituent or a different substituent.

Each R₅ variable may be independently selected from hydrogen, hydroxyl, an oxygen free radical, a hydroxyl amine of HTEMPO, a hydroxyl amine of acetate TEMPO, and a reduced sebacate TEMPO.

The polar aprotic solvent of the present disclosure is not particularly limited. In some embodiments, the polar aprotic solvent comprises a pKa of about 15 or more, such as about 15 to about 300, about 25 to about 300, about 35 to about 300, about 45 to about 300, about 55 to about 300, about 65 to about 300, about 75 to about 300, about 85 to about 300, about 95 to about 300, about 100 to about 300, about 200 to about 300, about 15 to about 100, about 15 to about 80, about 15 to about 60, about 30 to about 80, or about 30 to about 60. In this context, pKa refers to the pKa value of the most acidic proton in the solvent.

In some embodiments, the polar aprotic solvent comprises a dielectric constant of about 30 or more, such as about 30 to about 100, about 30 to about 90, about 30 to about 80, about 30 to about 70, about 30 to about 60, about 30 to about 50, or about 30 to about 40.

In other embodiments, the polar aprotic solvent comprises a dielectric constant of about 5 to about 50, such as about 5 to about 40 about 5 to about 30, about 5 to about 20, about 20 to about 50, or about 30 to about 40.

Illustrative, non-limiting examples of polar aprotic solvents include furfural, N-methyl-2-pyrrolidone (NMP), dimethylformamide (DMF), dimethylsulphoxide (DMSO), tetrahydrofuran (THF), acetonitrile,

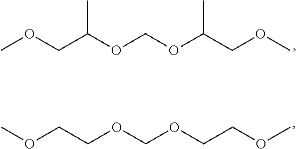

and any combination thereof.

In some embodiments, the solvent excludes non-polar solvents and/or polar protic solvents.

A composition of the present disclosure may include any amount of the polymerization inhibitor and the solvent. For example, a composition may comprise from about 1 wt. % to about 50 wt. % of the polymerization inhibitor, such as about 1 wt. % to about 40 wt. %, about 1 wt. % to about 30 wt. %, about 1 wt. % to about 20 wt. %, about 1 wt. % to about 10 wt. %, about 1 wt. % to about 5 wt. %, about 10 wt. % to about 50 wt. %, about 10 wt. % to about 20 wt. %, or about 15 wt. % to about 35 wt. % of the polymerization inhibitor.

A composition of the present disclosure may comprise, for example, from about 50 wt. % to about 99 wt. % of the polar aprotic solvent, such as from about 60 wt. % to about 99 wt. %, about 70 wt. % to about 99 wt. %, about 80 wt. % to about 99 wt. %, about 90 wt. % to about 99 wt. %, about 60 wt. % to about 90 wt. %, or about 70 wt. % to about 90 wt. % of the polar aprotic solvent.

The compositions of the present disclosure may include (or exclude) one or more additional components (other than the polymerization inhibitor and/or polar aprotic solvent) and/or one or more additional components may be added to the medium before, after, and/or with the polymerization inhibitor and/or the solvent.

Illustrative, non-limiting examples of additional components include a corrosion inhibitor, a dispersant, an additional solvent, a passivator, or any combination thereof.

The corrosion inhibitor may comprise, for example, an imidazoline compound, a pyridinium compound, a quaternary ammonium compound, a phosphate ester, an amine, an amide, a carboxylic acid, a thiol, and any combination thereof.

The dispersant may comprise, for example, a methacrylate, a polyisobutylene succinic anhydride polymer, a tall oil fatty acid ester, a tetraethylenepentamine tall oil fatty acid, a soy oil fatty acid, a soy oil fatty acid ester, a coconut fatty acid ester, and any combination thereof.

In some embodiments, the dispersant is selected from a polyolefin polyamine, magnesium carbonate, an alkyl magnesium sulfonic acid magnesium salt, maleic anhydride with a C₂₄-C₂₈ alpha-olefin polymer, an alpha-olefin C₂₄-C₂₈ succinamide polymer), a polyolefin ester, an alkenyl succinimide in mineral oil, and any combination thereof.

The additional solvent may comprise, for example, a heavy aromatic naphtha, kerosene, a light aromatic (e.g., benzene, toluene, ethylbenzene, xylene) a glycol, an ether, a glycol ether, and any combination thereof.

The passivator may comprise, for example, phosphoric acid, mono(2-ethylhexyl) ester, a compound comprising C₁₂-C₁₄ tert-alkylamines, formaldehyde, a polymer comprising one or more of 4-(1,1-dimethylethyl)-1,2-benzenediol, dodecylphenol, and/or 1,2-ethanediamine, and any combination thereof.

The additional component may be added to the medium before, after, and/or with the polar aprotic solvent and/or polymerization inhibitor. In some embodiments, the additional component is added with the polymerization inhibitor. In some embodiments, the additional component is added with the polar aprotic solvent. In some embodiments, the additional component is added separately (before and/or after) from the polymerization inhibitor. In some embodiments, the additional component is added separately (before and/or after) from the polar aprotic solvent. In some embodiments, the polymerization inhibitor and the polar aprotic solvent are added separately.

The amount of additional component added to the medium is not particularly limited. For example, from about 1 ppm to about 5,000 ppm of the additional component may be added to the medium, such as about 1 ppm to about 2,500 ppm, about 1 ppm to about 2,000 ppm, about 1 ppm to about 1,500 ppm, about 1 ppm to about 1,000 ppm, about 1 ppm to about 750 ppm, about 1 ppm to about 500 ppm, about 1 ppm to about 250 ppm, about 1 ppm to about 100 ppm, about 25 ppm to about 5,000 ppm, about 25 ppm to about 2,500 ppm, about 25 ppm to about 1,500 ppm, about 25 ppm to about 1,000 ppm, or about 25 ppm to about 500 ppm.

If a composition of the present disclosure comprises the additional component, the composition may comprise from, for example, about 0.1 wt. % to about 50 wt. % of the component, such as from about 0.1 wt. % to about 25 wt. %, about 0.1 wt. % to about 15 wt. %, about 0.1 wt. % to about 10 wt. %, about 0.1 wt. % to about 5 wt. %, about 1 wt. % to about 5 wt. %, about 1 wt. % to about 10 wt. %, about 1 wt. % to about 15 wt. %, about 5 wt. % to about 15 wt. %, about 5 wt. % to about 20 wt. %, about 10 wt. % to about 30 wt. %, or about 15 wt. % to about 25 wt. %.

In an illustrative embodiment, a composition of the present disclosure may comprise a polymerization inhibitor, a polar aprotic solvent, and an additional component, wherein the additional component comprises an additional solvent. The additional solvent may comprise, for example, toluene, the polar aprotic solvent may comprise, for example, NMP, and the polymerization inhibitor may comprise, for example, a compound of the following formula:

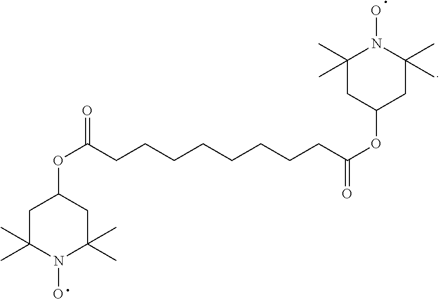

A composition may comprise from, for example, about 1 wt. % to about 10 wt. %, such as about 5 wt. %, of the polymerization inhibitor, about 15 wt. % to about 25 wt. %, such as about 20 wt. %, of the additional solvent, and about 70 wt. % to about 80 wt. %, such as about 75 wt. %, of the polar aprotic solvent.

In accordance with the present disclosure, various methods are disclosed for inhibiting the polymerization of monomers, such as ethylenically unsaturated monomers. Ethylenically unsaturated monomers are well-known in the art and all ethylenically unsaturated monomers are intended to be covered by the present disclosure.

In some aspects, the ethylenically unsaturated monomer is selected from the group consisting of acrylic acid, methacrylic acid, acrylonitrile, methacrylonitrile, acrolein, methacrolein, acrylate, methacrylate, acrylamide, methacrylamide, vinyl acetate, butadiene, ethylene, propylene, and styrene.

In some aspects, the compositions and methods disclosed herein are capable of inhibiting polymerization of a monomer selected from the groups consisting of chloroprene, acrylic acid, methacrylic acid, acrylonitrile, methacrylonitrile, methyl methacrylate, acrolein, methacrolein, acrylate, methacrylate, acrylamide, methacrylamide, vinyl acetate, butadiene, styrene, ethylene, propylene, ethylene dichloride, vinyl chloride, and any combination thereof.

Methods of the present disclosure include adding an effective amount of a composition disclosed herein to a medium comprising a reactive monomer and inhibiting polymerization of the monomer. Polymerization may be inhibited by, for example, capturing carbon centered radicals formed thermally by antipolymerant chemistry (e.g., polymerization inhibitors). The composition comprises a polymerization inhibitor as described herein and a polar aprotic solvent as described herein.

The medium comprising the reactive/ethylenically unsaturated monomer is not particularly limited and the methods and compositions disclosed herein can inhibit polymerization of the monomer regardless of the type of medium containing the monomer. As illustrative, non-limiting examples, a medium may comprise a fluid from the monomer manufacturing process, a fluid from a direct chlorination unit, a fluid from an oxychlorination unit, a fluid from a cracking unit, and any combination thereof.

In some embodiments, the medium may comprise a member selected from the group consisting of HCl, VCM, ethylene chloride, dichl-ethylene, chloroprene, 11-EDC, CHCl₃, CCl₄, C₆H₆, EDC, trichloroethylene, 112-trichl, heavy ends (which comprises a mixture of iron, coke, and tar—the tar comprising chlorinated hydrocarbons, such as 1,1,2-trichloroethylene), C₂H₂, benzene, and any combination thereof.

In certain instances, the medium may be an acidic medium comprising one or more acids and/or one or more acidic byproducts. Illustrative examples include hydrochloric acid, hypochlorous acid, chloric acid, perchloric acid, and any combination thereof. Additional examples include

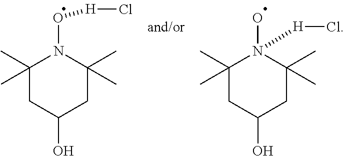

A polymerization inhibitor of the present disclosure may react with the acid in the medium and form a reaction product. The polar aprotic solvent may be useful for solubilizing the reaction product.

The amount of polymerization inhibitor added to the medium is not particularly limited and may be selected based on various factors, such as the amount of reactive monomer present in the medium. In some embodiments, the effective amount of polymerization inhibitor added to the medium is from about 1 ppm to about 10,000 ppm by weight of the ethylenically unsaturated monomer. Illustrative, non-limiting examples include about 1 ppm to about 5,000 ppm, about 1 ppm to about 2,000 ppm, about 1 ppm to about 1,000 ppm, about 1 ppm to about 800 ppm, about 1 ppm to about 600 ppm, about 1 ppm to about 400 ppm, about 1 ppm to about 250 ppm, about 1 ppm to about 200 ppm, about 1 ppm to about 100 ppm, about 1 ppm to about 75 ppm, about 1 ppm to about 50 ppm, about 1 ppm to about 25 ppm, about 10 ppm to about 100 ppm, or about 25 ppm to about 75 ppm of polymerization inhibitor added by weight of the ethylenically unsaturated monomer.

The compositions, compounds, and/or components disclosed herein may be added to the medium using a variety of different application methods known in the art. In some embodiments, the compositions, compounds, and/or components may added continuously or intermittently to the medium, either automatically or manually, by using, for example, chemical injection pumps. The chemical injection pumps may be programmed to add particular amounts of the compositions, compounds, and/or components at certain time intervals to the medium. In some aspects, the chemical injection pumps can be manually controlled to add particular amounts of the compositions, compounds, and/or components to the medium. In some embodiments, the addition may involve dripping, pouring, spraying, pumping, injecting, and/or otherwise adding the composition, compound, and/or component to the medium.

Any of the foregoing application methods may be independently selected to add a particular composition, compound, and/or component to the medium. Moreover, any of the forgoing application methods may be used to add a polymerization inhibitor, a polar aprotic solvent, and/or a component to the medium together or separately. For example, a chemical injection pump may be used to add a composition comprising a polymerization inhibitor and a polar aprotic solvent to the medium and a separate chemical injection pump may be used to add an additional component, such as a corrosion inhibitor, to the medium. As an additional example, the polymerization inhibitor may be added via a first chemical injection pump, the polar aprotic solvent may be added via a second chemical injection pump, and an additional component may be added via a third chemical injection pump.

In some embodiments, a composition of the present disclosure may be added to a medium during a vinyl chloride monomer manufacturing process. In the production of VCM, ethylene, oxygen and HCl are reacted in a direct chlorination or an oxychlorination unit to produce EDC, which is then processed in a cracking unit to form the VCM. The plant normally includes recycling facilities to recover additional EDC from the bottoms of the various units, thereby increasing total EDC recovery.

Significant fouling occurs in the various units handling the liquid EDC. For example, in the primary EDC recovery unit, fouling occurs in the distillation trays and the transfer facilities, particularly the retort furnace. EDC fouling is particularly serious in the liquid phase of EDC in the primary EDC recovery unit, the EDC recovery tar still, and the EDC recycle tar still. The fouling is believed to be due to highly chlorinated and/or oxygenated polymeric materials, which are incompatible in the EDC stream. It is not uncommon for the fouling to require plant shutdown after only a few days of operation. The presently disclosed compositions and methods can minimize or prevent fouling in any of the foregoing locations. The compositions of the present disclosure may be added before, after, and/or to any of the foregoing locations.

The foregoing may be better understood by reference to the following examples, which are intended for illustrative purposes and are not intended to limit the scope of the disclosure or its application in any way.

### Examples

A deposit comprising a foulant was obtained from an EDC unit. Deposit analysis revealed that the foulant contained degradation and/or byproducts of a prior art polymerization inhibitor composition along with corrosion byproducts. The prior art polymerization inhibitor composition included HTEMPO in butyl carbitol or xylene, which are non-polar solvents. These foulants caused under deposit corrosion, which further exasperated the fouling that led to corrosion and injection line blockage. The mechanism of formation of the deposit is hypothesized to be the protonation of HTEMPO by HCl present in the injection location, which is insoluble in the prior art solvents. Additional tests confirmed that the addition of acid to the prior art polymerization inhibitor composition caused the immediate formation of deposits.

The inventors hypothesized that utilizing a polymerization inhibitor and a polar aprotic solvent, such as NMP, may help keep the degradation products in solution as they are formed at the injection site. Indeed, testing onsite of HTEMPO and NMP revealed that the switch from a non-polar solvent to a polar aprotic solvent prevented the formation of solids upon introduction of HCl. The NMP kept the reaction byproducts in solution, thereby preventing precipitation. The formulations/compositions that were tested can be found in Table 1.

TABLE 1

| Inhibitor | Butylcarbitol | Dimethylphthalate | Aromatic (Solvesso 150) | NMP | DMF | DMSO | TOU | Toluene |
|---|---|---|---|---|---|---|---|---|
| HTEMPO | Deposits | NA | Deposits | No Deposits | No Deposits | No Deposits | Deposits | NA |
| Bis HTEMPO | NA | NA | NA | No Deposits | NA | NA | NA | Deposits |
| Sebacate | | | | | | | | |
| HTEMPO | NA | Deposits | NA | No Deposits | NA | NA | NA | Deposits |
| Acetate | | | | | | | | |

Testing revealed that only the compositions containing polar aprotic solvents did not produce deposits after the addition of acid to the composition. Furthermore, compositions containing, for example, Bis HTEMPO sebacate in toluene and HTEMPO in Aromatic 150 produced deposits upon acidification. Also HTEMPO in Elcosol DM produced deposits when an acid was added thereto. This shows that the combination of an inhibitor and polar aprotic solvent is a significant improvement over the prior art.

To carry out the foregoing experiments, 1 gram of polymerization inhibitor was dissolved in 9 grams of NMP, DMSO, and Elcosol, respectively, in a 10 mL test tube. To each tube was added either 100 uL (10,000 ppm) or 10 uL (1,000 ppm) of 37% HCl. The solutions were than monitored for any changes, such as discoloration, gelling, and the formation of deposits. Observations were made immediately after addition HCl, at 1 hr, and at 24 hours. The test tube with Elcosol contained visible amounts of deposits. The tubes containing NMP and DMSO were free of deposits and the solutions remained clear and homogenous.

The Elcosol-containing test tube was then decanted, leaving only the deposited solids. To the solids was added 10 mL of NMP. The test tube was shaken slightly for 5 seconds and observed. The deposits dissolved completely, leaving a homogenous solution, thereby proving the solubility hypothesis as the primary mechanism of removal of the byproducts.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a polymerization inhibitor” is intended to include “at least one polymerization inhibitor” or “one or more polymerization inhibitors.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25 °C with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of inhibiting polymerization of an ethylenically unsaturated monomer in a medium, comprising:
adding an effective amount of a composition to the medium, the composition comprising a polymerization inhibitor and a polar aprotic solvent, wherein the polymerization inhibitor comprises (4-hydroxy-2,2,6,6-tetramethylpiperidin-1-yl)oxyl (HTEMPO), a derivative thereof, and/or a hydroxylamine thereof, and
inhibiting polymerization of the monomer.

2. The method of claim 1, wherein the polymerization inhibitor comprises the following structure: 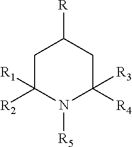
wherein each R₁, R₂, R₃ and R₄ is independently selected from a substituted or unsubstituted C₁-C₅₀ alkyl, alkenyl, or alkynyl group,
wherein R is selected from the group consisting of hydrogen, oxygen, alkyl, hydroxyl, alkoxyl, amino, N-alkylamino, amido, N-alkylamido, carboxylate and carboxyl, and
wherein R₅ is selected from hydrogen, hydroxyl, an oxygen free radical, a hydroxyl amine of HTEMPO, a hydroxyl amine of acetate TEMPO, and a reduced sebacate TEMPO.

3. The method of claim 1, wherein the polymerization inhibitor is selected from the group consisting of 4-hydroxy-2,2,6,6-tetramethyl piperidinol, 4-oxo-2,2,6,6-tetramethyl piperidinol, 4-methoxy-2,2,6,6-tetramethyl piperidinol, 4-acetate-2,2,6,6-tetramethyl piperidinol, 4-amino-2,2,6,6-tetramethyl piperidinol, 4-acetamido-2,2,6,6-tetramethyl piperidinol, 1,2,3,6-tetrahydro-2,2,6,6-tetramethyl piperidinol, bis(2,2,6,6-tetramethyl piperidinol) sebacate, and any combination thereof.

4. The method of claim 1, wherein the polymerization inhibitor is selected from the group consisting of 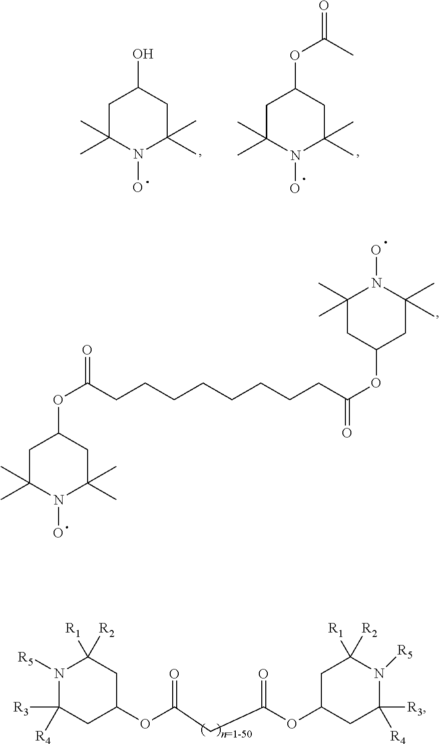
and any combination thereof,
wherein each R₁, R₂, R₃ and R₄ is independently selected from a substituted or unsubstituted C₁-C₅₀ alkyl, alkenyl, or alkynyl group, and
wherein each R₅ is independently selected from hydrogen, hydroxyl, an oxygen free radical, a hydroxyl amine of HTEMPO, a hydroxyl amine of acetate TEMPO, and a reduced sebacate TEMPO.

5. The method of claim 1, wherein the polar aprotic solvent comprises a pKa of about 15 or more and/or a dielectric constant of about 30 or more.

6. The method of claim 1, wherein the polar aprotic solvent is selected from the group consisting of furfural, N-methyl-2-pyrrolidone (NMP), dimethylformamide (DMF), dimethylsulphoxide (DMSO), tetrahydrofuran (THF), acetonitrile, 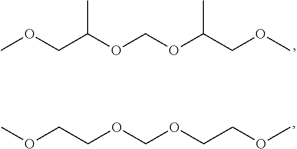
and any combination thereof.

7. The method of claim 1, further comprising adding the composition during a vinyl chloride monomer manufacturing process.

8. The method of claim 1, wherein the medium further comprises an acid.

9. The method of claim 8, further comprising reacting the acid with the polymerization inhibitor, forming a reaction product, and solubilizing the reaction product in the polar aprotic solvent.

10. The method of claim 1, wherein the composition comprises from about 1 wt. % to about 50 wt. % of the polymerization inhibitor and from about 50 wt. % to about 99 wt. % of the polar aprotic solvent.

11. The method of claim 1, wherein the ethylenically unsaturated monomer is selected from the group consisting of chloroprene, acrylic acid, methacrylic acid, acrylonitrile, methacrylonitrile, methyl methacrylate, acrolein, methacrolein, acrylate, methacrylate, acrylamide, methacrylamide, vinyl acetate, butadiene, styrene, ethylene, propylene, ethylene dichloride, vinyl chloride, and any combination thereof.

12. The method of claim 1, further comprising adding the composition prior to or in an ethylene dichloride purification unit.

13. The method of claim 1, wherein the composition comprises an additional component selected from the group consisting of a corrosion inhibitor, a dispersant, an additional solvent, a passivator, and any combination thereof.

14. The method of claim 13, wherein the additional solvent comprises toluene, the polar aprotic solvent comprises NMP, and the polymerization inhibitor comprises a compound of the following formula:

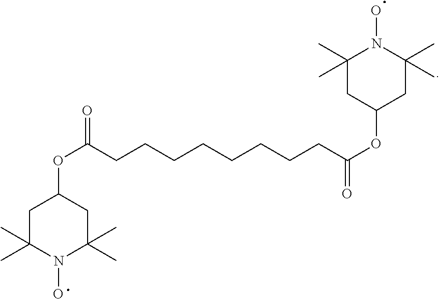

15. A solution, comprising:
a polymerization inhibitor, a polar aprotic solvent, and a reaction product of an acid and the polymerization inhibitor, wherein the polymerization inhibitor comprises HTEMPO, a derivative thereof, and/or a hydroxylamine thereof.

16. The solution of claim 15, wherein the polymerization inhibitor comprises the following structure: 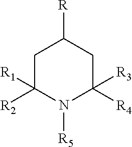
wherein each R₁, R₂, R₃ and R₄ is independently selected from a substituted or unsubstituted C₁-C₅₀ alkyl, alkenyl, or alkynyl group,
wherein R is selected from the group consisting of hydrogen, oxygen, alkyl, hydroxyl, alkoxyl, amino, N-alkylamino, amido, N-alkylamido, carboxylate and carboxyl, and
wherein R₅ is selected from hydrogen, hydroxyl, an oxygen free radical, a hydroxyl amine of HTEMPO, a hydroxyl amine of acetate TEMPO, and a reduced sebacate TEMPO.

17. The solution of claim 15, wherein the polymerization inhibitor is selected from the group consisting of 4-hydroxy-2,2,6,6-tetramethyl piperidinol, 4-oxo-2,2,6,6-tetramethyl piperidinol, 4-methoxy-2,2,6,6-tetramethyl piperidinol, 4-acetate-2,2,6,6-tetramethyl piperidinol, 4-amino-2,2,6,6-tetramethyl piperidinol, 4-acetamido-2,2,6,6-tetramethyl piperidinol, 1,2,3,6-tetrahydro-2,2,6,6-tetramethyl piperidinol, bis(2,2,6,6-tetramethyl piperidinol) sebacate, and any combination thereof.

18. The solution of claim 15, wherein the polymerization inhibitor is selected from the group consisting of 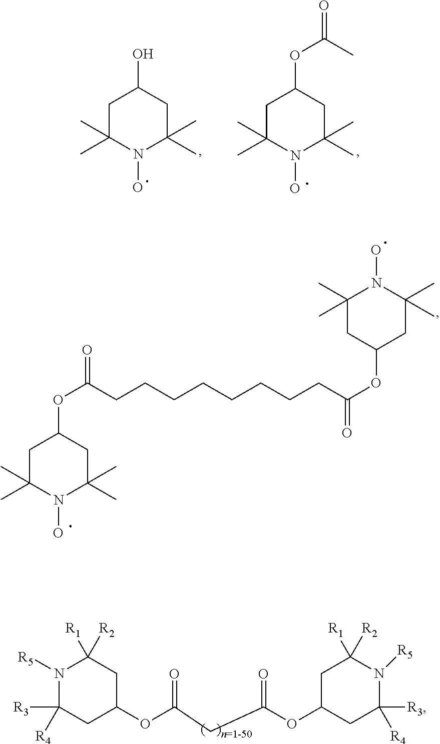
and any combination thereof,
wherein each R₁, R₂, R₃ and R₄ is independently selected from a substituted or unsubstituted C₁-C₅₀ alkyl, alkenyl, or alkynyl group, and
wherein each R₅ is independently selected from hydrogen, hydroxyl, an oxygen free radical, a hydroxyl amine of HTEMPO, a hydroxyl amine of acetate TEMPO, and a reduced sebacate TEMPO.

19. The solution of claim 15, wherein the polar aprotic solvent is selected from the group consisting of furfural, NMP, dimethylformamide (DMF), dimethylsulphoxide (DMSO), tetrahydrofuran (THF), acetonitrile, 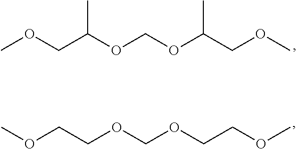
and any combination thereof.

20. The solution of claim 15, further comprising toluene, wherein the polar aprotic solvent comprises NMP and the polymerization inhibitor comprises a compound of the following formula: [chemical structure]
